(19) United States
(12) Patent Application Publication
(10) Pub. No.: US 2025/0367167 A1
(43) Pub. Date: Dec. 4, 2025

(54) COMPOSITION FOR MITOCHONDRIAL MODIFICATION COMPRISING A COMBINATION OF TWO OR MORE NATURAL PIGMENTS

(71) Applicant: University of Seoul Industry Cooperation Foundation, Seoul (KR)

(72) Inventors: Inhee CHOI, Seoul (KR); Hakchun Kim KIM, Seoul (KR)

(73) Assignee: University of Seoul Industry Cooperation Foundation, Seoul (KR)

(21) Appl. No.: 19/204,324

(22) Filed: May 9, 2025

(30) Foreign Application Priority Data
May 30, 2024 (KR) 10-2024-0070977

(51) Int. Cl. A61K 31/409; A61K 31/015; A61K 31/352; A61P 43/00

(52) U.S. Cl. CPC A61K 31/409; A61K 31/015; A61K 31/352; A61P 43/00

(57) ABSTRACT

The present invention relates to the control and enhancement of energy production of mitochondria through maximizing electron transfer efficiency using natural pigments and light. The composition for mitochondrial modification according to the present invention, which comprises two or more natural pigments selected from the group consisting of □-carotene, anthocyanin, and chlorophyll a as active ingredients, and the mitochondria modified by mixing these active ingredients have the effects of increasing ATP production and decreasing ROS production, and thus can be usefully used as a health functional food composition for improving exercise function and fatigue. Moreover, the effect of promoting the production of mitochondrial energy and preventing oxidative damage by light irradiation, can be used for the prevention and treatment of diseases related to mitochondrial dysfunction.

### CROSS-REFERENCE TO RELATED APPLICATIONS

This patent application claims the benefit of priority from Korean Patent Application No. 10-2024-0070977, filed on May 30, 2024, the contents of which are incorporated herein by reference.

### BACKGROUND OF THE INVENTION

### 1. Field of the Invention

The present disclosure relates to a composition for mitochondrial modification including a combination of two or more natural pigments, in which mitochondria modified with two or more natural pigments not only have enhanced resistance to oxidative stress, but also can enhance energy production by maximizing electron transfer efficiency by light irradiation.

### 2. Description of the Related Art

Mitochondria are organelles that perform metabolic functions within cells and produce adenosine triphosphate (ATP), which is used as an energy source for various cellular activities. Since ATP produced in mitochondria supplies about 90% of the metabolic energy required for eukaryotic cells, mitochondria are known as the powerhouse of the cell.

ATP production occurs in the electron transport chain present in the inner mitochondrial membrane. The electron transport chain consists of five protein complexes, I to V, each of which transfers electrons with high energy levels to the next complex, thereby ultimately producing ATP in complex V using the proton gradient formed in the inner mitochondrial membrane.

However, in the process of oxidative phosphorylation for ATP production, superoxide free radicals are essentially generated, and these oxygen radicals are converted into other reactive oxygen species (ROS), such as hydrogen peroxide and hydroxyl radicals.

These ROS cause oxidation of biopolymers such as lipids, proteins, and nucleic acids, impair mitochondrial function, and cause oxidative stress in cells.

Cells have antioxidant mechanisms to respond to such oxidative stress, and mitochondria damaged by ROS are mainly removed by autophagy, but external influences (e.g., aging and stress) decrease the antioxidant efficiency within the cells and increase the number of dysfunctional mitochondria.

As a result, oxidative stress caused by ROS reduces the ATP production capacity of mitochondria, which soon leads to the loss of cellular function, thereby resulting in cell senescence and cell death.

In fact, mitochondrial dysfunction is known to be the cause of various diseases, such as degenerative brain diseases (e.g., aging, cardiovascular disease, Alzheimer's disease, and Parkinson's disease), metabolic diseases (e.g., type 2 diabetes and obesity), and cancer.

Therefore, research and development are being conducted to treat or prevent various diseases by restoring mitochondrial function, reducing oxidative stress, and enhancing energy production.

The most widely attempted method to prevent oxidative damage to mitochondria is to administer antioxidants. To date, research results have been reported that various types of substances such as vitamin C, tocopherol, and plant-derived polyphenols can act as antioxidants to improve mitochondrial function, and methods have been prepared to utilize them for the prevention and treatment of diseases associated with mitochondrial dysfunction.

However, most of these antioxidants are structurally unstable, have low bioavailability, and merely function as radical scavengers, but do not provide the effect of directly promoting mitochondrial energy production or a means to reversibly induce the same externally.

Representatively, Korean Patent No. 10-0974202 discloses that a mitochondrial-targeting antioxidant compound can reduce oxidative stress within mitochondria by delivering a highly-concentrated antioxidant within mitochondria, but showed limitations in increasing mitochondrial metabolic activity itself or fundamentally strengthening mitochondrial function.

In addition, various methods have been proposed to resolve mitochondrial dysfunction, such as increasing ATP production by providing a substrate for ATP production, but these methods are limited in that they do not directly resolve mitochondrial energy production and reduction of oxidative damage.

Accordingly, there is a need for the development of a method for improving mitochondrial function that can be applied to the prevention and treatment of mitochondrial dysfunction diseases by directly controlling mitochondrial activity to increase energy production capacity while reducing ROS generation.

A recent study reported a method to increase mitochondrial membrane potential, oxygen consumption, and ATP production by enhancing mitochondrial electron transport chain activity using gold nanoparticles as efficient electron transport mediators (Nano Lett., 22 (19): 7927-7935, 2022).

Natural pigments contained in plants act as antennas that absorb photons during the photosynthetic process and cause electron transfer. Plants contain a variety of natural pigments to effectively receive light in the wavelength range ranging from red light to blue light.

Beta (β)-carotene is a natural pigment of the carotenoid series and is mainly found in green-yellow vegetables, such as carrots, old pumpkins, bell peppers, perilla leaves, lettuce, garlic chives, and spinach.

Anthocyanin, which is a polyphenol contained in berry fruits, is known as a natural pigment with high antioxidant activity.

Chlorophyll a, which is a natural pigment present in plant leaves, vegetable algae, or seaweed, is known to have a wide range of effects, including anticancer and anti-inflammatory effects, regenerating damaged cells, detoxification, and anti-cholesterol effects.

The above-mentioned natural pigments are widely used as food additives due to their antioxidant effects that can prevent damage caused by oxidation in animal tissues, their high nutritional values, and their unique colors, and they are also useful as antioxidants related to skin aging. In addition, much research is being conducted on treatment methods utilizing the anticancer effects of natural pigments, and their effects in improving cardiovascular diseases, degenerative brain diseases, and inflammation.

Research has also been conducted on techniques to increase mitochondrial activity by utilizing the above-mentioned natural pigments, but the use of each natural pigment alone showed an insignificant effect on promoting ATP production by light irradiation, and there were limitations in fundamentally enhancing mitochondrial function by simultaneously removing ROS (Agnieszka Sliwa et al., Acta Biochim Pol., 2012; Jing Li et al., J Funct Foods, 2023; Chen Xu et al., J Cell Sci, 2014).

Under the circumstances, the inventors of the present invention have attempted to develop a technology to overcome the limitations of prior art and directly increase mitochondrial activity and prevent oxidative damage. As a result, they have discovered that by modifying mitochondria with a combination of two or more natural pigments, ROS production can be reduced while simultaneously increasing ATP production through light irradiation, thereby completing the present invention.

### SUMMARY OF THE INVENTION

One object of the present invention is to provide a composition that can increase the resistance of mitochondria to oxidative stress and enhance functions such as ATP production using natural pigments.

Another object of the present invention is to provide mitochondria with enhanced function by modifying them with natural pigments.

In order to achieve the above-mentioned objects, an aspect of the present invention provides a composition for mitochondrial modification, which includes two or more natural pigments selected from the group consisting of carotene, anthocyanin, and chlorophyll as active ingredients.

In addition, an aspect of the present invention provides a method for preparing modified mitochondria, which includes mixing the composition for mitochondrial modification and isolated mitochondria.

In addition, an aspect of the present invention provides mitochondria modified by the above-mentioned preparation method.

In addition, an aspect of the present invention provides a health functional food composition for enhancing mitochondrial function, which includes two or more natural pigments selected from the group consisting of β-carotene, anthocyanins, and chlorophyll a as active ingredients.

### BRIEF DESCRIPTION OF THE DRAWINGS

The above and other objects, features and other advantages of the present invention will be more clearly understood from the following detailed description taken in conjunction with the accompanying drawings, in which:

FIGS. 1A and 1B shows a schematic diagram and an image illustrating the process of isolating mitochondria from cells and the results of fluorescent staining of the isolated mitochondria using MitoTracker Deep Red, which is a mitochondrial-specific fluorescent marker;

FIG. 1C to 1E shows images illustrating the surface of mitochondria observed through scanning electron microscope image analysis.

FIG. 1F shows an image illustrating the internal structure of mitochondria observed through transmission electron microscope image analysis;

FIGS. 1G and 1H shows an image and a graph illustrating the measurement results of the topology and height profile of mitochondria using atomic force microscope image analysis;

FIG. 11 shows the measurement results of the surface potential of mitochondria;

FIG. 2A to 2D shows the results of calculated predicted values and actual measured values as absorption spectra according to combinations of natural pigments;

FIG. 2E to 2H shows the results of analyzing the absorption spectrum over time after mixing a combination of natural pigments;

FIG. 3 shows the measurement results of the amount of ATP produced by mitochondria including anthocyanins according to light irradiation time;

FIG. 4 shows the measurement results of mitochondrial ATP production according to combinations of natural pigments and light wavelengths, illustrating the calculated predicted values and actual measured values;

FIG. 5 shows a schematic diagram illustrating the electron transport process for ATP production, in mitochondria including any combination of three natural pigments, according to light irradiation;

FIG. 6 shows the measurement results of the amount of ATP production according to the increase in the concentration of mitochondria including a combination of three natural pigments;

FIG. 7 shows a graph illustrating the amounts of ATP and ROS produced by mitochondria according to a combination of natural pigments; and

FIGS. 8A and 8B shows the measurement results of the amount of ATP produced when light was irradiated after treatment with inhibitors for each protein complex that constitutes the mitochondrial electron transport chain.

### DETAILED DESCRIPTION OF THE INVENTION

Hereinafter, the present invention will be described in detail.

Unless otherwise defined, all technical and scientific terms used herein have the same meaning as commonly understood by one of ordinary skill in the art to which the present invention belongs. The terminology used in the description of the present invention is only for the purpose of effectively describing specific Examples and Experimental Examples and is not intended to limit the present invention.

Furthermore, throughout the specification, an element “including” means that, unless otherwise specifically stated, it may further include other elements, rather than excluding other elements.

An aspect of the present invention relates to a composition for mitochondrial modification, which includes two or more natural pigments selected from the group consisting of carotene, anthocyanin, and chlorophyll as active ingredients.

The natural pigments used in the present invention may be those obtained by extracting, separating, and purifying according to a method known in the art from a plant including the corresponding natural pigments, or synthesized pigments may be used. Natural pigments may be used after the purification to a purity acceptable for food science or pharmaceutical purposes, or by purchasing those commercially available. As a specific method for obtaining natural pigments from plants, an extraction method using water, or alcohols in which alcohol and acids are added may be used, and separation and purification methods using ion column chromatography, preparative thin-layer chromatography, preparative high performance liquid chromatography, liquid-liquid partition extraction, precipitation, etc. may be used.

The chlorophyll is a green pigment involved in photosynthesis and is found in plants, algae, and cyanobacteria. It absorbs blue and red light well, and chlorophyll a and chlorophyll b are found in green plants, chlorophyll c1, chlorophyll c2, chlorophyll e are found in algae, and chlorophyll d and chlorophyll f are known to exist in cyanobacteria. In the present invention, chlorophyll includes all of these chlorophylls, preferably includes chlorophyll a and chlorophyll b, and more preferably includes chlorophyll a. Chlorophyll may be used in the form of a mixture of two or more chlorophylls.

The carotene, which is a type of carotenoid, includes alpha-carotene, beta-carotene, gamma-carotene, delta-carotene, etc. Beta-carotene is known to exist most abundantly in plants as an antioxidant, and as a yellow or orange pigment. In the present invention, carotene includes all of the above-mentioned carotenes, but preferably includes beta-carotene. Carotene may be used in the form of a mixture of two or more carotenes.

The anthocyanin, which is a natural pigment derived from plants, may include one or more selected from the group consisting of peonidin, cyanidin 3-arabinoside, cyanidin-3-(xylosylglucose)-5-galactose, cyanidin 3-glucoside, cyanidin-3-xyloside, cyanidin 3-galactoside, cyanidin-3-(coumaroyl-xylosylglucose)-5-galactose, delphinidin 3-glucoside, delphinidin 3-rutinoside, peonidin 3-arabinoside, peonidin 3-galactoside, petunidin 3-glucoside, cyanidin, delphinidin, malvidin, pelargonidin, peonidin, cyanidin 3,5-diglucoside, cyanidin 3-rutinoside, pelargonidin 3-glucoside, peonidin 3-glucoside, malvidin 3-glucoside, and malvidin 3,5-diglucoside, and preferably cyanidin-3-galactoside, cyanidin-3-glucoside, cyanidin-3-arabinoside, cyanidin-3-xyloside, etc.

In the case of anthocyanins, they may be used in a form that includes a mixture of pigments rather than specific pigment compounds. A mixture of extracted anthocyanins may be used depending on the plant from which anthocyanins are isolated.

In the present invention, the composition for mitochondrial modification is for the purpose of modifying mitochondria, and in the present invention, modification includes binding of a natural pigment to mitochondria, and the binding includes all bindings in which the pigment penetrates into the inside of the mitochondria or binds from the outside.

Pigments can bind to the outer membrane of mitochondria, can penetrate mitochondria and bind to the intermembrane space, can bind to the inner membrane, or can bind to the matrix of mitochondria.

The composition for mitochondrial modification modifies mitochondria by binding to them. Meanwhile, by irradiating light on the modified mitochondria, the ROS production in mitochondria is reduced, and the electron transfer efficiency of mitochondria is maximized, thereby synergistically increasing ATP production.

When combining two or more pigments, a combination in which the respective band gaps partially overlap may be used. In the combination of pigments having partially overlapping band gaps, excited (photoinduced) electrons are transferred from a pigment with a high LUMO energy level to another pigment with a lower LUMO energy level, and the electrons are transferred from the combined pigments to the electron transport chain related to ATP production in mitochondria, thereby synergistically increasing electron transfer and consequently synergistically increasing ATP production efficiency compared to the case where a single pigment is used.

For example, as schematically presented in FIG. 5, when there are arbitrary pigments X, Y, and Z having energy band gaps that are different from one another but at least partially overlapping, electrons are sequentially transferred from the LUMO energy level of pigment X to pigment Y and to pigment Z, and the electrons transferred in this way flow into the electron transport chain of ATP production.

In order to obtain maximum efficiency in the process of exciting and/or emitting electrons contained in the natural pigments, each of the natural pigments may be irradiated with light of a different wavelength.

In the present invention, regarding a combination of two or more natural pigments, when light is irradiated at wavelengths where the maximum absorption ranges of the natural pigments overlap, electron transfer efficiency may be maximized through an interaction of electrons contained in the natural pigments. Once the maximum absorption wavelength of each pigment is determined, the wavelength range of light to be irradiated may be determined to include wavelengths of −50 nm to +50 nm, −40 nm to +40 nm, −30 nm to +30 nm, −20 nm to +20 nm, or −10 nm to +10 nm centered on the maximum absorption wavelength of the pigment used for modification.

Even when two or more pigments are mixed, the wavelength and range of light to be irradiated may be determined by being centered on the maximum absorption wavelength by measuring the absorption wavelength regarding the mixed pigments, and when two or more absorption wavelength peaks appear, the wavelength and range of light to be irradiated may be determined by being centered on each of the two or more absorption wavelength peaks.

When two or more pigments are mixed, the absorption wavelength peak appears at the absorption wavelength peak possessed by each pigment, but the peak height ratio of each absorption wavelength peak may vary depending on the mixing ratio.

According to a specific Experimental Example of the present invention, in the case of β-carotene, light in a wavelength range of 410-510 nm, preferably 430-490 nm, and more preferably 450-480 nm may be irradiated.

According to a specific Experimental Example of the present invention, in the case of anthocyanins, light in a wavelength range of 500-600 nm, preferably 520-580 nm, and more preferably 540-560 nm may be irradiated.

According to a specific Experimental Example of the present invention, in the case of chlorophyll a, light in a wavelength range of 360-460 nm or 610-710 nm, preferably 380-440 nm or 630-690 nm, more preferably 400-420 nm or 650-670 nm may be irradiated.

According to a specific Experimental Example of the present invention, in the case of a natural pigment combination composed of β-carotene and anthocyanin, light in a wavelength range of 410-600 nm, preferably 450-550 nm, may be irradiated.

According to a specific Experimental Example of the present invention, in the case of a natural pigment combination composed of β-carotene and chlorophyll a, light in a wavelength range of 360-710 nm, preferably 570-680 nm, may be irradiated.

According to a specific Experimental Example of the present invention, in the case of a natural pigment combination composed of anthocyanins and chlorophyll a, light having a wavelength range of 500-710 nm, preferably 600-700 nm, and more preferably 640-680 nm can be irradiated.

According to a specific Experimental Example of the present invention, in the case of a natural pigment combination composed of β-carotene, anthocyanin, and chlorophyll a, light in a wavelength range of 410-710 nm, preferably 570-700 nm, more preferably 640-680 nm may be irradiated.

In the present invention, the time for irradiating light may be 0-120 minutes, preferably 40-100 minutes, and more preferably 70-90 minutes.

In the present invention, the intensity of light may be 1-10 mW, preferably 3-8 mW, and more preferably 4-6 mW.

In the present invention, an LED may be used as a light source.

The composition for mitochondrial modification according to the present invention may increase ATP production of mitochondria by way of light irradiation.

The composition for mitochondrial modification may include β-carotene, anthocyanin, and chlorophyll a.

In a preferred Example of the present invention, the composition for mitochondrial modification to increase ATP production of mitochondria may include β-carotene, anthocyanin, and chlorophyll a in a ratio of 5:2 to 4:1 to 3.

In a preferred Example of the present invention, the composition for mitochondrial modification may increase mitochondrial ATP production by up to 45-fold by way of light irradiation.

In the present invention, the amount of ATP production in mitochondria may be measured using a method known in the art.

The composition for mitochondrial modification according to the present invention may reduce ROS production in mitochondria by way of light irradiation.

In the present invention, the amount of ROS produced in mitochondria may be measured using a method known in the art.

In a preferred example of the present invention, the composition for mitochondrial modification may comprise β-carotene and chlorophyll a in a ratio of 5:1 to 3 so as to reduce the ROS production by mitochondria.

Another aspect of the present invention provides a method for preparing modified mitochondria, which includes mixing a composition for mitochondrial modification and isolated mitochondria.

In the present invention, the composition for mitochondrial modification solution including a combination of two or more natural pigments may be mixed with isolated mitochondria. The solution may be stirred to promote binding of the natural pigments to the mitochondria.

In the present invention, the step of mixing the composition for mitochondrial modification and isolated mitochondria is not particularly limited, but may be performed at room temperature or lower, and in one specific embodiment, may be performed at 0-10° C.

In the present invention, the step of mixing a composition for mitochondrial modification and isolated mitochondria may be performed for a time sufficient to form a sufficient binding, for example, for 6 hours or more (overnight).

In the present invention, modified mitochondria may be obtained by centrifuging a mixture of the composition for mitochondrial modification and isolated mitochondria followed by resuspension of the sedimented precipitant.

In the present invention, the method for preparing modified mitochondria may further include isolating mitochondria from an individual.

The individual may be a mammal including a human.

The step of isolating mitochondria from an individual may use a method for obtaining mitochondria from cells known in the art.

The cells may be one or more types of cells selected from the group consisting of fibroblasts, muscle cells, hepatocytes, epithelial cells, endothelial cells, nerve cells, adipocytes, bone cells, leukocytes, lymphocytes, stem cells, and mucosal cells.

Still another aspect of the present invention provides mitochondria modified by the method for preparing mitochondria. The modification is as described above.

In the present invention, the composition for mitochondrial modification or modified, to which a composition for mitochondrial modification is bound, have the effect of promoting the production of mitochondrial energy and preventing oxidative damage by light irradiation, and thus can be used for the prevention and treatment of diseases related to mitochondrial dysfunction.

The diseases related to mitochondrial dysfunction may include aging diseases, cardiovascular diseases, Alzheimer's disease, and Parkinson's disease.

Still another aspect of the present invention provides a health functional food composition for enhancing mitochondrial function, which includes two or more natural pigments selected from the group consisting of β-carotene, anthocyanin, and chlorophyll a as active ingredients.

The natural pigments can increase resistance to oxidative stress by reducing ROS production in mitochondria by binding to mitochondria, and can also maximize electron transfer efficiency within mitochondria to thereby synergistically increase ATP production. Meanwhile, it is obvious to those skilled in the art that light must be appropriately irradiated for this purpose, and the above-mentioned description applies as-is with respect to the appropriate wavelength of light.

In the present invention, the composition can achieve improvement in fatigue by enhancing mitochondrial function.

In the present invention, the composition can achieve improved exercise capacity by enhancing mitochondrial function.

Hereinafter, the present invention will be described in detail through Examples and Experimental Examples.

### <Example 1> Isolation of Mitochondria

### 1-1. Mitochondria-Specific Fluorescent Labeling

In order to isolate mitochondria from human dermal fibroblasts (HDFs), a mitochondrial isolation kit for cultured cells (Thermo Fisher Scientific, Cat. No. 89874) was used.

Isolated mitochondria were immersed in the Dulbecco's modified Eagle medium (DMEM) solution including mitochondrial labeling probes.

As the mitochondrial labeling probe, MitoTracer Deep Red (Thermo Fisher Scientific, Cat. No. M22426) was used at 100 nM.

The isolated mitochondria and probe were reacted at 4° C. for 30 minutes, and then centrifuged at 12,000×g for 15 minutes.

Fluorescently labeled mitochondria were resuspended in DMEM and then observed using a confocal microscope (LSM 800, Carl Zeiss).

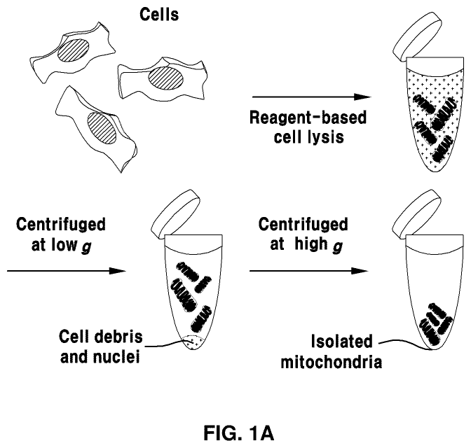

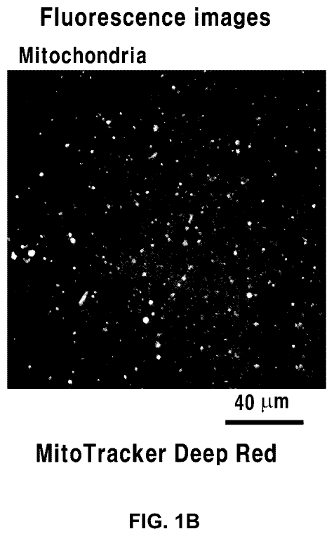

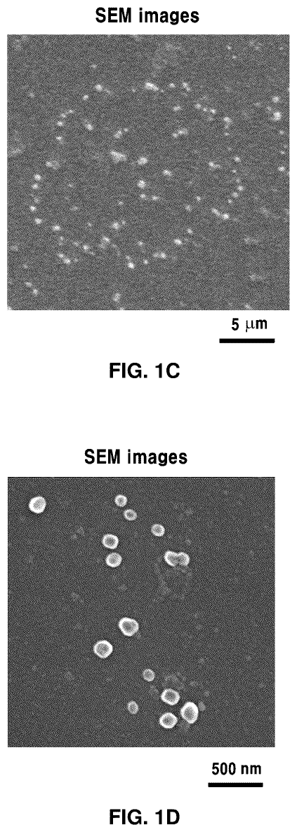

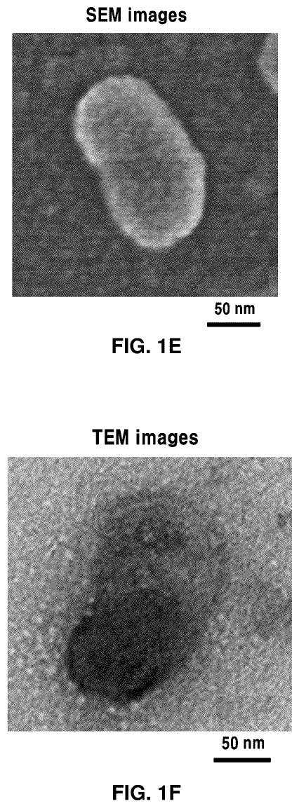

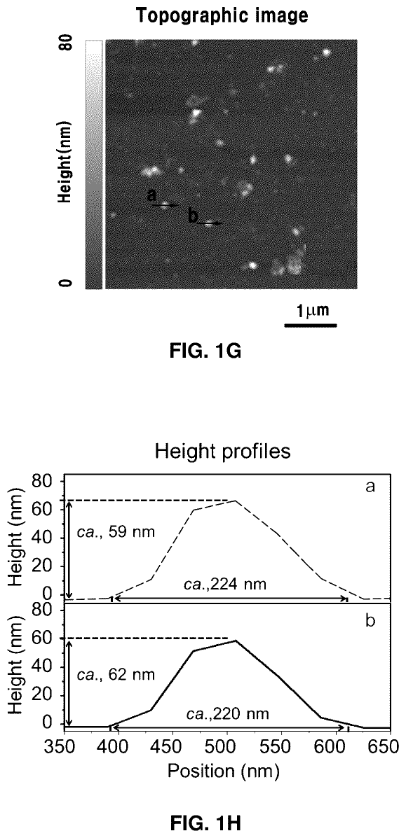

The mitochondria isolation process and the results of fluorescent labeling analysis are shown in FIGS. 1A and 1B.

By obtaining and analyzing the fluorescence images thereof, it was confirmed that the labeled mitochondria exhibited stable and distinct fluorescence.

### 1-2. Analysis of Images of Scanning Electron Microscopy (SEM)

Mitochondria were isolated from HDF cells using the same method as in <Example 1-1>.

The analysis of scanning electron microscope images was performed to observe the surface of mitochondria.

The surface morphology of mitochondria was confirmed using a field emission scanning electron microscope (SU8010, HITACHI) at an accelerating voltage of 10 kV.

The analysis of the surface images of mitochondria confirmed that the size of mitochondria is about 50 nm in the short axis and 200 nm in the long axis (see FIG. 1C to 1E).

### 1-3. Analysis of Images of Transmission Electron Microscopy (TEM)

The analysis of the TEM images was performed to characterize the isolated mitochondria.

In order to prepare samples for analysis, 10 μL of mitochondrial pellet in phosphate buffered saline (PBS) was dropped onto a carbon-coated 300-mesh TEM grid (Ted Pella, Inc.).

TEM images were obtained using a transmission electron microscope (LIBRA 120, Carl Zeiss) operating at an accelerating voltage of 120 kV.

The analysis of TEM images of mitochondria confirmed that the sizes were about 50 nm in the short axis and 200 nm in the long axis (see FIG. 1F).

### 1-4. Analysis of Images of Atomic Force Microscopy (ATM)

In order to analyze the surface topology and height profiles of isolated mitochondria, images were obtained in a non-contact mode using an atomic force microscope (NX12-bio, Park Systems).

The analysis of the surface topology images and height profiles of the mitochondria confirmed that the size of the isolated mitochondria was about 60 nm in height, specifically 59 nm or 62 nm, and that the long axis was about 200 nm, specifically 220 nm or 224 nm (see FIGS. 1G and 1H).

### 1-5. Measurement of Zeta Potential of Isolated Mitochondria

In order to measure the zeta potential of isolated mitochondria, the potential of the mitochondrial surface was analyzed using an electrophoretic light scattering spectrophotometer (ELS-Z2000, Otsuka Electronics).

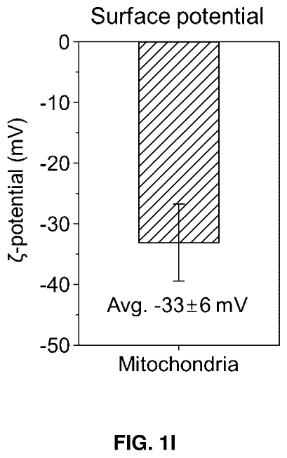

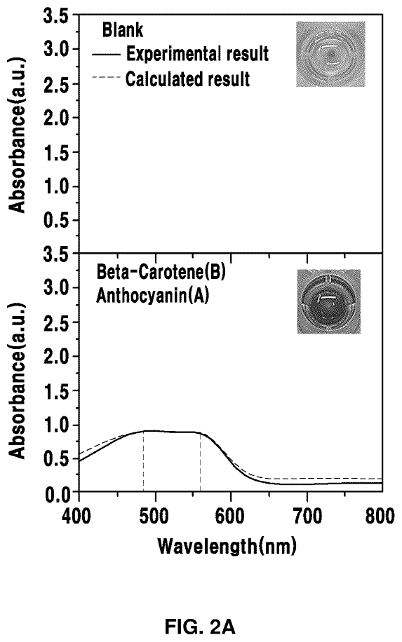

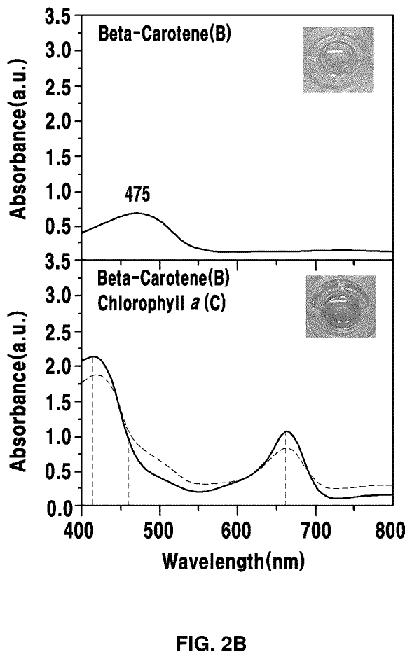

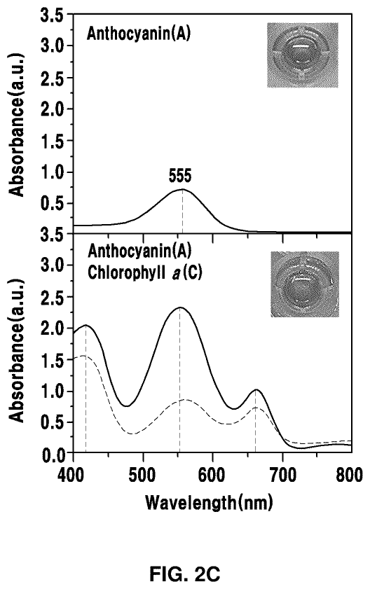

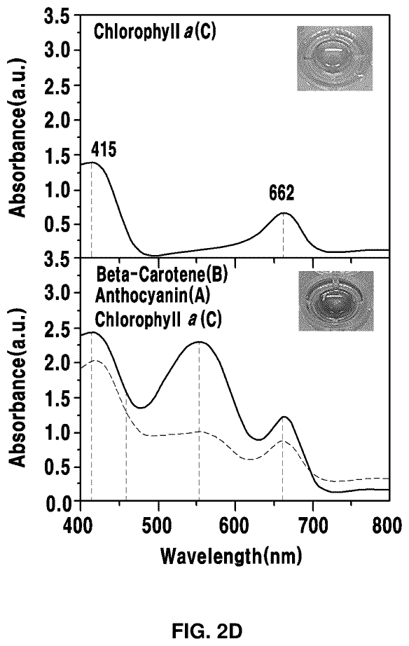

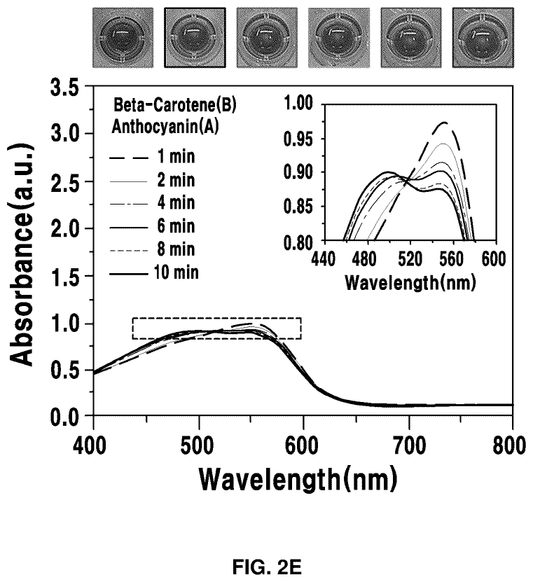

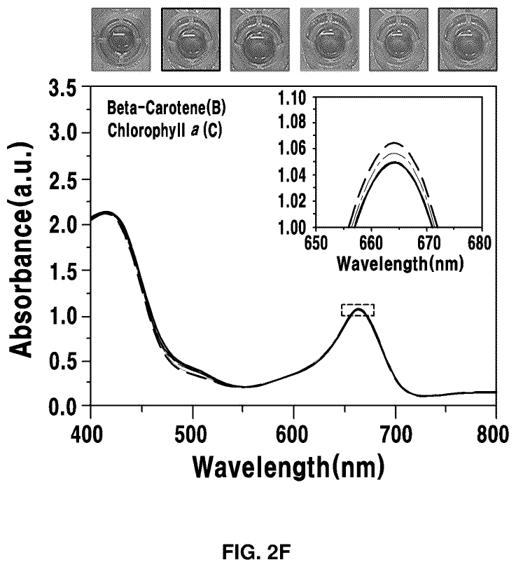

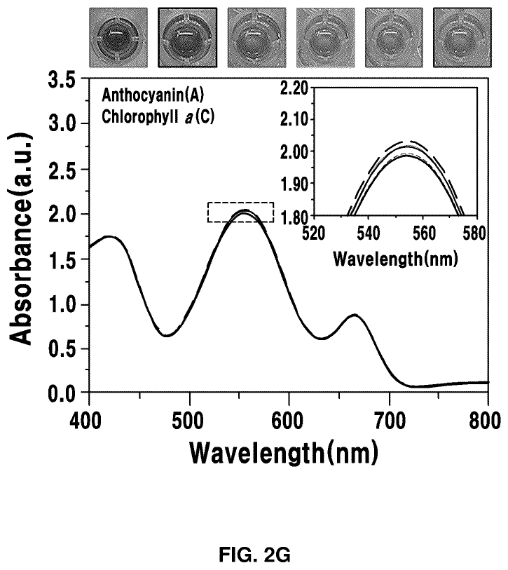

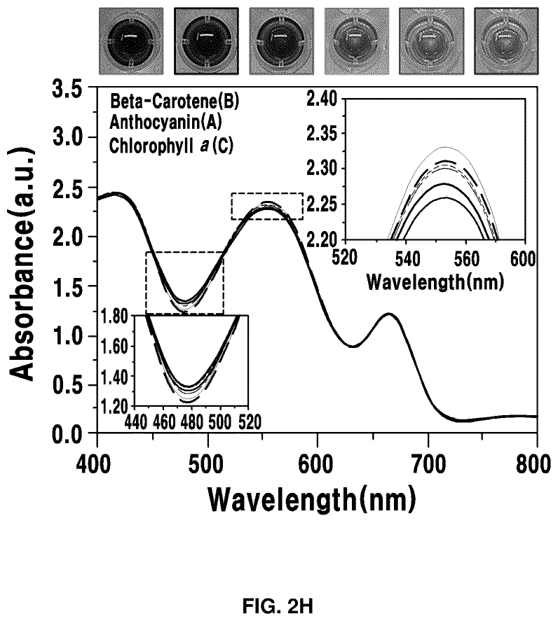

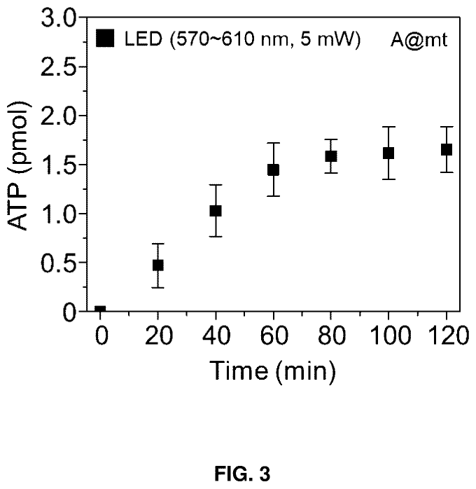

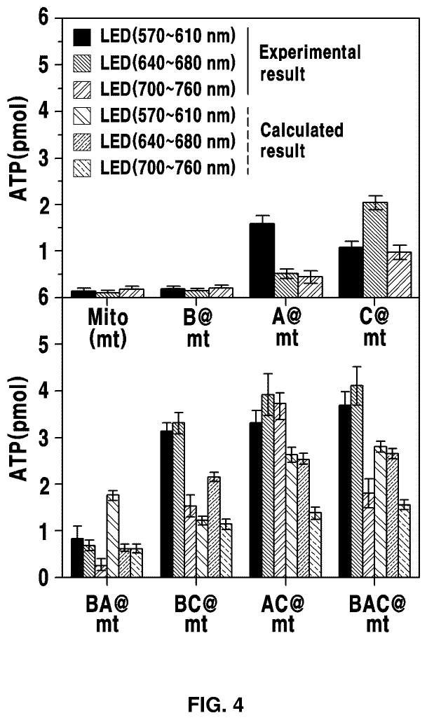

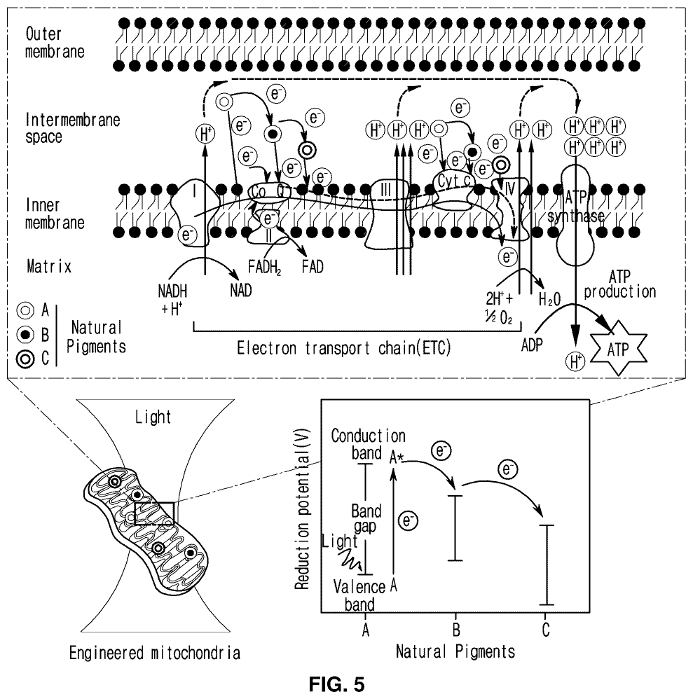

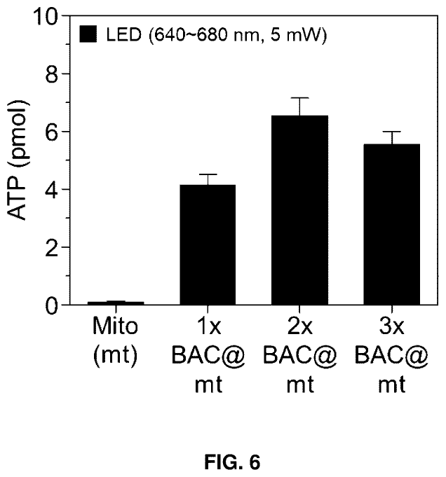

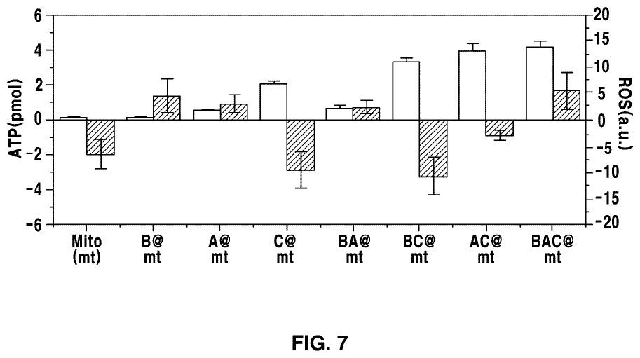

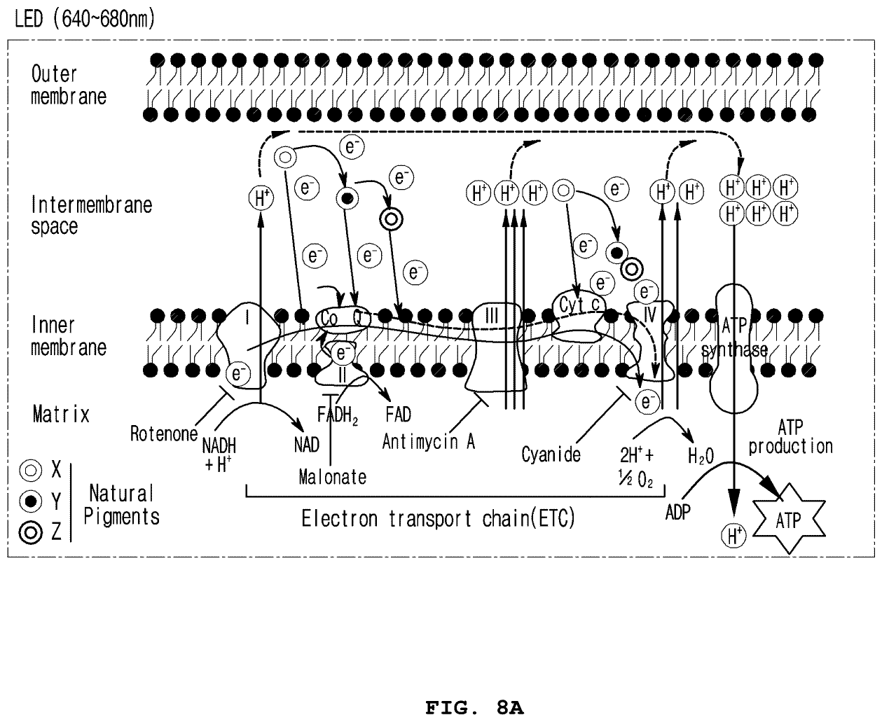

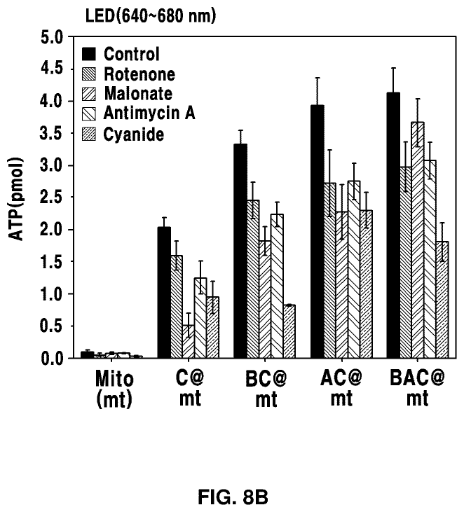

It was confirmed that the surface zeta potential of isolated mitochondria showed an average negative charge of −33±6 mV (see FIG. 11).

### <Example 2> Absorption Spectrum Analysis of Natural Pigments

In order to analyze the absorbance spectra of three natural pigments (i.e., β-carotene, delphinidin chloride (anthocyanin), and chlorophyll a (Sigma-Aldrich)), the absorbance analysis was performed using a UV/Vis NANO spectrophotometer (Nabi).

As a result of the absorption spectrum analysis of 100 μM β-carotene (labeled as “B” in the drawing), 60 UM anthocyanins (labeled as “A” in the drawing), and 40 μM chlorophyll a (labeled as “C” in the drawing), it was confirmed that β-carotene exhibited high absorbance at 475 nm, anthocyanins exhibited high absorbance at 555 nm, and chlorophyll a exhibited high absorbance at 415 nm and 662 nm (FIG. 2A to 2D).

In the drawing, “Experimental result” corresponds to the actually-measured absorption spectrum, and “Calculated result” represents the result of added-up values of the absorption spectrum of single natural pigments.

Referring to FIG. 2A to 2D, when combining two or more antenna pigments (i.e., combining β-carotene and chlorophyll a, anthocyanins and chlorophyll a, and β-carotene, anthocyanin, and chlorophyll a), the resulting values of the actual absorption spectra showed a difference from the cumulative values of the single pigments.

The results of the absorption spectrum analysis over time by mixing 100 UM B-carotene, 60 μM anthocyanin, and 40 μM chlorophyll a in various combinations are shown in FIG. 2E to 2H.

As a result, it was confirmed that a significant change occurred in the absorbance for 10 minutes, particularly under the condition where β-carotene and anthocyanins were mixed.

These results imply that changes in the absorption spectrum can be induced by combinations and interactions between natural pigments.

### <Experimental Example 1> Enhancement of Mitochondrial Function Using Natural Pigments and Measurement of ATP Production According to Light Irradiation Time

In order to enhance mitochondrial function using natural pigments, mitochondria (indicated as “mt” in the drawing) were isolated by the method according to <Example 1>, and 200 μL of anthocyanins (A) was mixed with 200 μL of the isolated mitochondria, and the mixture was cultured overnight at 4° C.

Mitochondria including anthocyanins (A@mt) were obtained by centrifugation at 12,000×g for 15 minutes followed by resuspension in PBS.

In order to confirm the effect of anthocyanins on promoting ATP production in mitochondria and to optimize light irradiation conditions for the same, the following experiments were conducted.

50 μL of mitochondria (A@mt) including anthocyanins were added to a 96-well black plate, and light with a wavelength of 570-610 nm, which is close to the maximum absorption wavelength of anthocyanins (555 nm), was irradiated at an intensity of 5 mW at 20 minutes intervals for 0-120 minutes.

After the light irradiation, the amount of ATP production according to the light irradiation time was measured using an ATP Assay Kit (Abcam).

An additional 50 μL of an ATP reaction mixture was added and incubated for 60 minutes at room temperature under a light-blocked condition.

Then, the amount of ATP production was measured by measuring the fluorescence intensity (Ex/Em=535/587 nm), and the results are shown in FIG. 3.

As a result, it was confirmed that ATP production gradually increased from 0 to 80 minutes and was maintained after 80 minutes.

These results indicate that the ATP production is increased by the interaction between light and natural pigments.

According to the results above, the light irradiation conditions were optimized by setting the intensity of the light irradiation on mitochondria including natural pigments to 5 mW, and the light irradiation time to 80 minutes.

### <Experimental Example 2> Modulation of Mitochondrial ATP Production by Combination of Natural Pigments and Light Wavelengths

In order to discover the optimal combination of natural pigments that enhance mitochondrial function, mitochondria (indicated as “mt” in the drawing) were isolated by the method according to <Example 1>, and 200 μL of the isolated mitochondria were mixed with 200 UL each of solutions of natural pigments β-carotene (B), anthocyanins (A), and chlorophyll a (C), either alone or in combination, and the mixtures were cultured overnight at 4° C.

The concentrations and combinations of natural pigments used to enhance mitochondrial function are shown in Table 1 below.

Mitochondria including each combination of natural pigments were obtained by centrifugation at 12,000×g for 15 minutes followed by resuspension in PBS.

TABLE 1

| | | | | | | | | | Distilled |
|---|---|---|---|---|---|---|---|---|---|
| | | Mitochondria | β-carotene | | Anthocyanins | | Chlorophyll a | | Water |
| Combination | | (mt) | (B) | | (A) | | (C) | | (D.W.) |
| 1 | Mito | 200 μL | — | | — | | — | | 200 μL |
| 2 | B@mt | 200 μL | 200 | μM | — | | — | | — |
| | | | 200 | μL | | | | | |
| 3 | A@mt | 200 μL | — | | 120 | μM | — | | — |
| | | | | | 200 | μL | | | |
| 4 | C@mt | 200 μL | — | | — | | 80 | μM | — |
| | | | | | | | 200 | μL | |
| 5 | BA@mt | 200 μL | 400 | μM | 240 | μM | — | | — |
| | | | 100 | μL | 100 | μL | | | |
| 6 | BC@mt | 200 μL | 400 | μM | — | | 160 | μM | — |
| | | | 100 | μL | | | 100 | μL | |
| 7 | AC@mt | 200 μL | — | | 240 | μM | 160 | μM | — |
| | | | | | 100 | μL | 100 | μL | |
| 8 | BAC@mt | 200 μL | 600 | μM | 360 | μM | 240 | μM | — |
| | | | 67 | μL | 67 | μL | 67 | μL | |

50 μL of mitochondria including each combination of natural pigments were added to a 96-well black plate, and irradiated with light of three different wavelength ranges (570-610 nm, 640-680 nm, and 700-760 nm) for 80 minutes.

After light irradiation, 50 μL of the ATP reaction mixture was additionally mixed to measure the amount of ATP production and the mixture was incubated at room temperature for 60 minutes under a light-blocked condition.

Then, the amount of ATP production was measured by measuring the fluorescence intensity (Ex/Em=535/587 nm) using a microplate reader.

The amount of ATP production was calculated by subtracting the fluorescence value measured upon irradiation with light for 0 minutes from the fluorescence value measured upon irradiation with light for 80 minutes, and the results are shown in FIG. 4.

As a result, it was confirmed that the amount of ATP production can be controlled depending on the combination of natural pigments and light wavelengths.

More specifically, it was confirmed that the mitochondria including a single natural pigment increased the amount of ATP production when irradiated with light of a wavelength close to the maximum absorption wavelength of the natural pigment.

Additionally, it was found that in the case of mitochondria including a combination of two or more natural pigments, most actually-measured ATP concentrations were higher than the simple added-up ATP concentration of the ATP concentrations of individual single pigments.

This suggests that electronic interactions between pigments in the combination of natural pigments may affect the amount of ATP produced by mitochondria.

Among them, the mitochondria (BAC@mt) including a combination of three natural pigments (i.e., β-carotene (B), anthocyanins (A), and chlorophyll a (C)) produced 4.1 pmol of ATP when irradiated with light of wavelength at 640-680 nm.

Under the wavelength conditions of 640-680 nm, the effect was confirmed in which the amount of ATP production increased up to 45-fold higher in mitochondria including the three natural pigment combinations compared to the control mitochondria (Mito) that did not include natural pigments, which produced 0.09 pmol of ATP.

These results suggest that the electrons of natural pigments, when irradiated with light of a wavelength that matches the absorption spectrum of natural pigments, become excited and participate in the functioning of the electron transport chain (ETC) of mitochondria, thereby enabling the control of ATP production (see FIG. 5).

The amount of ATP production according to the concentration of mitochondria was measured under the conditions of combinations of natural pigments and light wavelengths exhibiting the highest ATP production (i.e., in mitochondria which included all of β-carotene (B), anthocyanins (A), and chlorophyll a (C) and which were irradiated with light wavelengths of wavelengths of 640-680 nm) (see FIG. 6).

When the amount of mitochondria including a combination of the three natural pigments was 1-fold, about 4 pmol of ATP was produced; when the amount of mitochondria including a combination of the three natural pigments was doubled, about 6.5 pmol of ATP was produced; and when the amount of mitochondria including a combination of the three natural pigments was tripled, the amount of ATP production decreased to about 5.5 pmol.

These results imply that when the mitochondria including the combination of the three natural pigments exceed a certain concentration, a blocking effect occurs on the interaction between light and the mitochondria including the combination of the three natural pigments, thereby decreasing the amount of ATP production.

Therefore, these results indicate that the amount of ATP production by mitochondria can be controlled by controlling, in a combinatorial fashion, the types of natural pigments, the wavelength and irradiation time of the light being irradiated, and the concentration of mitochondria including the natural pigments.

### <Experimental Example 3> Measurement of Reactive Oxygen Species (ROS) Production According to Enhanced Mitochondrial Function Using Natural Pigments and Lights

It is generally known that ROS are generated from protein complexes involved in the electron transport process during oxidative phosphorylation in mitochondria.

Accordingly, in order to measure the amount of ROS produced by enhancing mitochondrial function using natural pigments, an analysis was performed using the Cellular ROS Assay Kit (Abcam).

50 μL of mitochondria including each combination of natural pigments were added to a 96-well black plate, and irradiated with light having a wavelength of 640-680 nm at an intensity of 5 mW for 80 minutes.

After light irradiation, 50 μL of mitochondria including each combination of natural pigments were transferred to a 1.5 mL tube, and 50 μL of 80 UM dichlorodihydrofluorescein diacetate (DCFDA) in 1× buffer was added to fluorescently stain the ROS produced, and the resultant was incubated at 4° C. for 30 minutes under light-blocking conditions.

For washing, mitochondria were centrifuged at 12,000×g for 15 minutes using 1× buffer, and the supernatant was removed, and the resultant was resuspended in 100 μL of 1× buffer.

Then, the amount of ROS production was measured by measuring the fluorescence intensity (Ex/Em=485/535 nm) using a microplate reader.

The amount of ATP production was calculated by subtracting the fluorescence value measured upon irradiation with light for 0 minutes from the fluorescence value measured upon irradiation with light for 80 minutes, and the results are shown in FIG. 7.

It was confirmed that ROS production decreased merely by irradiating the control mitochondria (Mito) that did not include natural pigments.

This shows the possibility that light irradiation can facilitate electron transfer, thereby inducing inhibition of ROS production.

Among the mitochondria including a single natural pigment or a combination of two or more natural pigments, the mitochondria including chlorophyll a alone (C@mt), a combination of β-carotene and chlorophyll a (BC@mt), and a combination of anthocyanins and chlorophyll a (AC@mt) showed a tendency that ROS production decreases upon light irradiation.

In particular, in the cases where mitochondria included only chlorophyll a (C@mt) and a combination of β-carotene and chlorophyll a (BC@mt), the amount of ATP production increased significantly and the amount of ROS production decreased in response to light irradiation, compared to the control mitochondria (Mito).

These results imply that there are optimal conditions that can decrease ROS production while simultaneously increasing ATP production.

In summary, the mitochondria including a combination of β-carotene and chlorophyll a, when irradiated with light having a wavelength of 640-680 nm at an intensity of 5 mW for 80 minutes, showed the best effect in terms of ATP production compared to ROS production in mitochondria.

### <Experimental Example 4> Evaluation of Amount of ATP Production According to Inhibitor Treatment for Each Protein Complex Constituting Mitochondrial Electron Transport Chain

It is generally known that ATP is produced through protein complexes involved in the electron transport process during the oxidative phosphorylation process of mitochondria. Accordingly, an analysis was performed using the ATP Assay Kit (Abcam) to evaluate the amount of ATP production when treating with inhibitors for each protein complex.

A total of 50 μL of mitochondria including each combination of natural pigments (C@mt, BC@mt, AC@mt, or BAC@mt) and 2 μM of inhibitors (Rotenone, Malonate, Antimycin A or Cyanide) for each protein complex were added to the E-tube and incubated at room temperature for 1 hour. Then, the supernatant was removed using a centrifuge (12,000 g, 15 minutes) and resuspended in 50 μL of PBS. 50 μL of mitochondria including a combination of natural pigments treated with inhibitors were added to each 96-well black plate, and irradiated with light having a wavelength of 640-680 nm at an intensity of 5 mW for 80 minutes.

After light irradiation, 50 μL of the ATP reaction mixture was additionally mixed so as to measure the amount of ATP production, and the mixture was incubated at room temperature for 60 minutes under a light-blocked condition.

Then, the amount of ATP production was measured by measuring the fluorescence intensity (Ex/Em=535/587 nm) using a microplate reader. The amount of ATP production was calculated by subtracting the fluorescence value measured upon irradiation with light for 0 minutes from the fluorescence value measured upon irradiation with light for 80 minutes, and the results are shown in FIGS. 8A and 8B.

As a result, the treatment with an inhibitor lead to a decrease of most of the ATP production in mitochondria although there were some differences, thus implying that the flow of electrons generated by the interaction between light and pigments is involved in the ETC channel. For example, the treatment with malonate under the C@mt condition lead to a rapid decrease ATP production, thus implying that many electrons of C may be transferred between complex I and complex II.

Mitochondria modified with a combination of two or more natural pigments according to the present invention not only increase resistance to oxidative stress by reducing ROS production, but also maximize electron transfer efficiency by light irradiation, thereby synergistically increasing ATP production.

The modified mitochondria according to the present invention have excellent energy production efficiency and can be utilized as a health functional food composition for improving exercise function and fatigue, and can be applied to the development of therapeutic agents for diseases related to mitochondrial dysfunction, such as aging, cardiovascular disease, Alzheimer's disease, and Parkinson's disease.

The Examples and Experimental Examples above are merely specific illustrations of the present invention in one aspect, and the present invention is not limited to these Examples and Experimental Examples. In other words, since the Examples and Experimental Examples presented can be modified in various ways, the scope of the present invention should be understood to include equivalents or substitutes capable of implementing the technical spirit of the present invention.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A composition for mitochondrial modification comprising two or more natural pigments selected from the group consisting of carotene, anthocyanins, and chlorophyll as active ingredients.

2. The composition as set forth in claim 1, wherein the composition increases production of adenosine triphosphate (ATP) in mitochondria by light irradiation.

3. The composition as set forth in claim 1, wherein the composition reduces production of reactive oxygen species (ROS) in mitochondria by light irradiation.

4. The composition as set forth in claim 1, wherein the carotene comprises β-carotene, the anthocyanins comprise delphinidin, and the chlorophyll comprises chlorophyll a.

5. A method for preparing modified mitochondria comprising mixing a composition for mitochondrial modification as set forth in claim 1 and isolated mitochondria.

6. The method as set forth in claim 5, further comprising isolating mitochondria from an individual.

7. Mitochondria modified by the method as set forth in claim 5.

8. A health functional food composition for enhancing mitochondrial function, comprising two or more natural pigments selected from the group consisting of β-carotene, anthocyanin, and chlorophyll a as active ingredients.

9. The health functional food composition as set forth in claim 8, wherein the composition achieves an improvement in fatigue by enhancing mitochondrial function.

10. The health functional food composition as set forth in claim 8, wherein the composition achieves an improvement in exercise capacity by enhancing mitochondrial function.
